(19) United States
(12) Patent Application Publication
(10) Pub. No.: US 2023/0149145 A1
(43) Pub. Date: May 18, 2023

(54) DUAL GEL COLORIMETRIC EFFERVESCENT DENTAL CALCULUS SOFTENING/TOOTH WHITENING/ ANTI-INFLAMMATORY CLEANING COMPOSITION WITH INSTANTANEOUS MIXING VIA DOUBLE BARREL SYRINGE DELIVERY

(71) Applicant: Essential Dental Systems, Inc., South Hackensack, NJ (US)

(72) Inventors: Jason Edward Guzman, Union City, NJ (US); Allan Deutsch, Boca Raton, FL (US)

(21) Appl. No.: 17/527,438

(22) Filed: Nov. 16, 2021

(51) Int. Cl. A61C 19/06; A61Q 11/00; A61C 17/00; A61K 8/04

(52) U.S. Cl. CPC A61C 19/063; A61Q 11/00; A61C 17/00; A61K 8/042; A61K 2800/884; A61K 2800/92

(57) ABSTRACT

A method includes placing a double barrel syringe into a dispensing gun. The method also includes using the dispensing gun to place a composition of paste A and paste B within the double barrel syringe through a mixing tip onto a tooth surface and interproximal areas along a gumline and/or subgingival areas. The composition softens and aids in removal of dental calculus, tarter, and/or plaque on one or more areas of the tooth surface that require cleaning. The method also includes rinsing the one or more areas of the tooth surface to enable the cleaning to begin.

### TECHNICAL FIELD

The present disclosure generally relates to the application of a composition onto a tooth surface, along the gumline, and interproximal areas in order to effectively clean as well as remove dental calculus/plaque/tartar.

### BACKGROUND

Mineralized plague and deep stains can be present on the teeth of patients. Moreover, in subgingival areas and interproximal areas along the gumline of patients, such hardened plaque, tartar, and other deep stains are often present.

In addition, a need exists to clean these areas thoroughly without harming areas along the gumline, subgingivally, and/or other areas along the dental arch and tooth surfaces. As such, cleaning needs to take place without causing excessive harm to the patient as a result of the more conventional dental cleaning methods such as manual or ultrasonic scaling. These conventional dental cleaning methods may result in tooth enamel erosion and excessive inflammation of the soft oral tissues.

Accordingly, a need exists for a cleaning composition that cleans the interproximal areas and or subgingivally areas. The composition should be able to greatly reduce the plaque, tartar, and other dental calculus that is present.

Further, a need exists for the cleaning composition to greatly reduce any dental calculus in these areas (tooth surface, gumline, subgingival areas) efficiently without causing any excess harm to the tooth surface, gumline, and/or subgingival areas of the patient.

### SUMMARY

The following summary is provided to facilitate an understanding of some of the features of the disclosed embodiments and is not intended to be a full description. A full appreciation of the various aspects of the embodiments disclosed herein can be gained by taking the specification, claims, drawings, and abstract as a whole.

The aforementioned aspects and other objectives can now be achieved as described herein.

In an embodiment, a method includes placing a double barrel syringe into a dispensing gun. The method also includes using the dispensing gun to place a composition of paste A and past B within the double barrel syringe onto a tooth surface. The composition softens and aids in removal of dental calculus on one or more areas of the tooth surface. The method also includes rinsing the one or more areas of the tooth surface to enable the cleaning to begin.

A combination of the composition softens the dental calculus in the one or more areas of the tooth surface to enable the cleaning to occur.

The method also includes reapplying the composition onto the tooth surface to provide additional cleaning for the tooth surface.

In an embodiment, a method includes placing composition in a double barrel syringe. The method also includes disposing the composition through a mixing tip configured at an end of the double barrel syringe onto a tooth surface to contact unclean areas of the tooth surface to soften dental calculus to enable the dental calculus to be more readily removed and enable the unclean areas of the tooth surface to be cleaned after a reaction time. The method also includes rinsing the tooth surface after the reaction time to enable the cleaning to occur on the unclean areas of the tooth surface.

The method also includes dispensing the composition through the mixing tip onto a dental tray.

The reaction time is less than or equal to ninety seconds.

In an embodiment, a system includes a double barrel syringe placed into a dispensing gun, wherein a mixing tip is affixed to the dispensing gun. The dispensing gun to place a composition composed of paste A and paste B within the double barrel syringe onto a tooth surface. The composition softens and aids in removal of dental calculus on one or more areas of the tooth surface that require cleaning. The one or more areas of the tooth surface rinsed to enable the cleaning to begin on the one or more areas of the tooth surface.

The composition includes an alkaline phase and an acidic phase.

The combined paste A and paste B loosens or softens the dental calculus to aid its removal so that less force is required during a scaling process.

### BRIEF DESCRIPTION OF THE FIGURES

The accompanying figures, in which like reference numerals refer to identical or functionally similar elements throughout the separate views and which are incorporated in and form a part of the specification, further illustrate the present invention and, together with the detailed description of the invention, serve to explain the principles of the present invention.

FIG. 1 is a diagram of pastes in accordance with an embodiment of the invention;

FIG. 2 illustrates a system in accordance with an embodiment of the invention;

FIG. 3(A) illustrates a system in accordance with an embodiment of the invention;

FIG. 3(B) illustrates another aspect of the system in accordance with an embodiment of the invention;

FIG. 4(A) illustrates a system in accordance with an embodiment of the invention;

FIG. 4(B) illustrates another version of the system in accordance with an embodiment of the invention;

FIG. 4(C) depicts another aspect of the system in accordance with an embodiment of the invention;

FIG. 5 illustrates a graph in accordance with an embodiment of the invention; and

FIG. 6 depicts a flow chart in accordance with an embodiment of the invention

Unless otherwise indicated illustrations in the figures are not necessarily drawn to scale.

### DETAILED DESCRIPTION OF SOME EMBODIMENTS

### Background and Context

The particular values and configurations discussed in these non-limiting examples can be varied and are cited merely to illustrate one or more embodiments and are not intended to limit the scope thereof.

Subject matter will now be described more fully herein after with reference to the accompanying drawings, which form a part hereof, and which show, by way of illustration, specific example embodiments. Subject matter may, however, be embodied in a variety of different form and, therefore, covered or claimed subject matter is intended to be construed as not being limited to any example embodiments set forth herein, example embodiments are provided merely to be illustrative. Likewise, a reasonably broad scope for claimed or covered subject matter is intended. Among other issues, subject matter may be embodied as methods, devices, components, or systems. The followed detailed description is, therefore, not intended to be interpreted in a limiting sense.

Throughout the specification and claims, terms may have nuanced meanings suggested or implied in context beyond an explicitly stated meaning. Likewise, phrases such as “in one embodiment” or “in an example embodiment” and variations thereof as utilized herein may not necessarily refer to the same embodiment and the phrase “in another embodiment” or “in another example embodiment” and variations thereof as utilized herein may or may not necessarily refer to a different embodiment. It is intended, for example, that claimed subject matter include combinations of example embodiments in whole or in part.

In general, terminology may be understood, at least in part, from usage in context. For example, terms such as “and,” “or,” or “and/or” as used herein may include a variety of meanings that may depend, at least in part, upon the context in which such terms are used. Generally, “or” if used to associate a list, such as A, B, or C, is intended to mean A, B, and C, here used in the inclusive sense, as well as A, B, or C, here used in the exclusive sense. In addition, the term “one or more” as used herein, depending at least in part upon context, may be used to describe any feature, structure, or characteristic in a singular sense or may be used to describe combinations of features, structures, or characteristics in a plural sense. Similarly, terms such as a “a,” “an,” or “the”, again, may be understood to convey a singular usage or to convey a plural usage, depending at least in part upon context. In addition, the term “based on” may be understood as not necessarily intended to convey an exclusive set of factors and may, instead, allow for existence of additional factors not necessarily expressly described, again, depending at least in part on context.

One having ordinary skill in the relevant art will readily recognize the subject matter disclosed herein can be practiced without one or more of the specific details or with other methods. In other instances, well-known structures or operations are not shown in detail to avoid obscuring certain aspects. This disclosure is not limited by the illustrated ordering of acts or events, as some acts may occur in different orders and/or concurrently with other acts or events. Furthermore, not all illustrated acts or events are required to implement a methodology in accordance with the embodiments disclosed herein.

Unless defined otherwise, all technical and scientific terms used herein have the same meanings as commonly understood by one of ordinary skill in the art to which the disclosed embodiments belong. Preferred methods, techniques, devices, and materials are described, although any methods, techniques, devices, or materials similar or equivalent to those described herein may be used in the practice or testing of the present invention.

Although claims have been included in this application to specific enumerated combinations of features, it should be understood the scope of the present disclosure also includes any novel feature or any novel combination of features disclosed herein.

References “an embodiment,” “example embodiment,” “various embodiments,” “some embodiments,” etc., may indicate that the embodiment(s) so described may include a particular feature, structure, or characteristic, but not every possible embodiment necessarily includes that particular feature, structure, or characteristic.

Headings provided are for convenience and are not to be taken as limiting the present disclosure in any way.

Each term utilized herein is to be given its broadest interpretation given the context in which that term is utilized.

### Terminology

The following paragraphs provide context for terms found in the present disclosure (including the claims):

The transitional term “comprising”, which is synonymous with “including,” “containing,” or “characterized by,” is inclusive or open-ended and does not exclude additional, unrecited elements or method steps. See, e.g., Mars Inc. v. H. J. Heinz Co., 377 F.3d 1369, 1376, 71 USPQ2d 1837, 1843 (Fed. Cir. 2004) (“[L]ike the term ‘comprising,’ the terms ‘containing’ and ‘mixture’ are open-ended.”). “Configured to” or “operable for” is used to connote structure by indicating that the mechanisms/units/components include structure that performs the task or tasks during operation. “Configured to” may include adapting a manufacturing process to fabricate components that are adapted to implement or perform one or more tasks.

“Based On.” As used herein, this term is used to describe factors that affect a determination without otherwise precluding other or additional factors that may affect that determination. More particularly, such a determination may be solely “based on” those factors or based, at least in part, on those factors.

All terms of example language (e.g., including, without limitation, “such as”, “like”, “for example”, “for instance”, “similar to”, etc.) are not exclusive of other examples and therefore mean “by way of example, and not limitation . . . ”.

A description of an embodiment having components in communication with each other does not infer that all enumerated components are needed.

A commercial implementation in accordance with the scope and spirit of the present disclosure may be configured according to the needs of the particular application, whereby any function of the teachings related to any described embodiment of the present invention may be suitably changed by those skilled in the art.

The flowchart and block diagrams in the figures illustrate the architecture, functionality, and operation of possible implementations of systems and methods according to various embodiments. Functions noted in the block may occur out of the order noted in the figures. For example, two blocks shown in succession may, in fact, be executed substantially concurrently, or the blocks may sometimes be executed in the reverse order, depending upon the functionality involved.

Further, any sequence of steps that may be described does not necessarily indicate a condition that the steps be performed in that order. Some steps may be performed simultaneously.

The functionality and/or the features of a particular component may be alternatively embodied by one or more other devices that are not explicitly described as having such functionality/features. Also, various embodiments of the present invention need not include a device itself.

More specifically, as will be appreciated by one skilled in the art, aspects of the present invention may be embodied as a system and/or method. Furthermore, aspects of the present invention may take the form of a plurality of systems to enable gas meter to perform self-checking to determine its overall functioning without requiring a meter operator.

### INTRODUCTION

Embodiments of the present invention include two different pastes, paste A and paste B, which are placed into a double barrel syringe. The double barrel syringe each includes a barrel for paste A and paste B respectively. Paste A and Paste B will be place in their respective barrel within the double barrel syringe. A mixing tip will be attached to the double barrel syringe. The paste A and paste B will be combined within the mixing tip in to a single mixed composition to be used to clean the tooth surface, interproximal areas, and subgingival areas of any dental calculus, tartar, plaque, etc.

The double barrel syringe and the mixing tip are configured within a dispensing gun. The dispensing gun can then dispense the mixing composition through the mixing tip onto a dental tray. The dental tray can be used to place the mixed composition on the tooth surface of a patient, including the interproximal areas along the gumline and subgingival areas.

The dental tray will be filled with the mixed composition, and the dental tray with the mixed composition will be placed over the dental arch of the patient. The tooth surface, interproximal area along the gumline, and subgingival areas will be above the dental arch. The mixed composition will come into contact with the dental calculus in and around the tooth surface, gumline, and subgingival areas. The mixed composition will soften and loosen the dental calculus including tarter, plaque and deep stains. Moreover, the mixed composition will remain on the tooth surface, interproximal areas along the gumline, and subgingival areas for a reaction time from thirty to ninety seconds. As the reaction time increases, the micro-hardness of the dental calculus is reduced.

After the reaction time is complete, rinsing liquid such as water will rinse the mixed composition off of the tooth surface, interproximal areas, and subgingival areas. Rinsing off the mixed composition will enable the cleaning to begin. The dental calculus, plaque, tarter, and other deep stains can be greatly reduced as a result. In addition, if additional cleanings are required, the mixed composition can be placed again on the tooth surface, interproximal areas, and subgingivally areas.

### System Structure

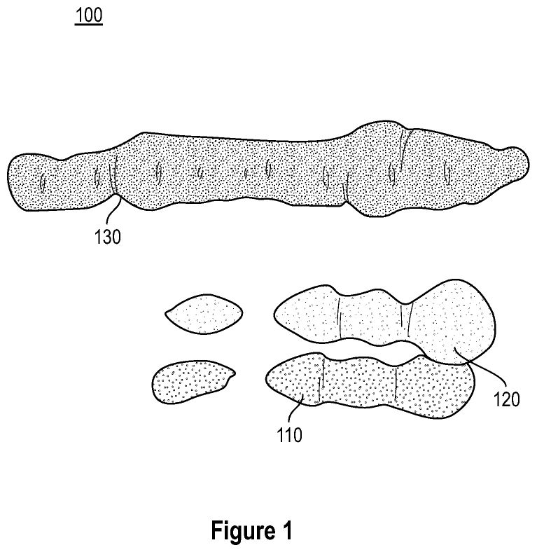

In FIG. 1, a system. 1.00 is illustrated in which the composition used to loosen the dental calculus on the tooth surface is shown. Paste A 110 and a paste B are 120 illustrated. The paste A 110 and the paste B are placed into the double barrel syringe. The mixing tip is affixed to an end of the double barrel syringe. When the paste A 110 and the paste B 120 is dispensed through the mixing tip, they are mixed together and combined into the composition that is shown. The different colors of paste A 110 and paste B 120 respectively will cause the combined colorization of the composition when paste A 110 and paste B 1.20 are mixed together thru the mixing tip as the mixed composition 130 is disposed on the tooth surface. The mixed composition 130 will include a seafoam green colorization that will contact the dental calculus on the tooth surface, interproximal areas, and subgingivally areas of the one or more persons. The mixed composition 130 with the colorization will be set on the tooth surface for a reaction time. The reaction time can be at least thirty seconds, In addition, the reaction time can last as long as ninety seconds or more.

Referring to FIG. 1, as the mixed composition 130 is set on the tooth surface and other areas such as the interproximal areas and subgingivally areas for the reaction time, the mixed composition 130 is able to loosen and soften the dental calculus on the tooth surface. The hardened plaque and deep stains are loosed and softened. The mixed composition 130 will loosen and soften the dental calculus such as hardened mineralized plaque, tartar and other stains for the reaction time in which the mixed composition 130 remains on the tooth surface. The mixed composition 130 loosening the dental calculus will make it easier to remove the dental calculus and ensure that the tooth surface is clean when the tooth surface is rinsed with water or other rinsing fluids. Whether the reaction time is thirty seconds, or up to ninety seconds, the time that the mixed composition 130 is allowed to loosen the dental calculus aids in the removal of the dental calculus on the tooth surface, interproximal areas, and/or subgingivally areas on the dental arch. The rinsing fluid can rinse the mixed composition 130 off of the tooth surface to complete the cleaning. Further, the mixed composition 130 can also be placed back on the tooth surface to start another cleaning of the tooth surface if necessary.

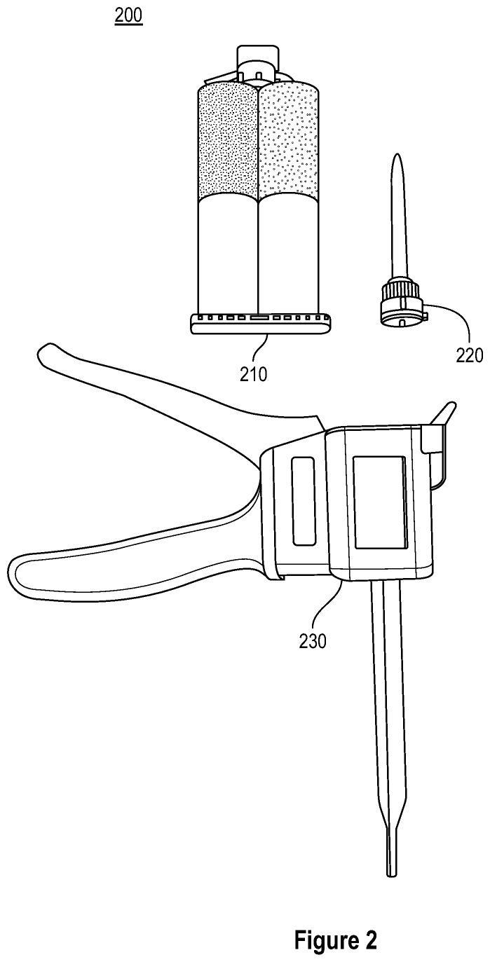

FIG. 2 illustrates a cleaning system 200 in accordance with an embodiment of the invention. The cleaning system 200 can be used to clean the surface of teeth including interproximal and/or subgingivally areas found in one or more people. The teeth of various individuals can include dental calculus that includes, but is not limited, hardened mineralized plaque and deep stains. The cleaning system 200 can enable the dental calculus to be removed or greatly reduced from the teeth of various individuals. The cleaning system 200 includes a double barrel syringe 210 that includes a paste A and a paste B, The paste A and the paste B can be in separate barrels within the double barrel syringe 210. The paste A and paste B can ultimately be combined into a single composition when applied on the teeth of one or more persons. A mixing tip 220 can be placed at the end of the double barrel syringe 110. The double barrel syringe 210 along with the mixing tip 220 can be placed within a dispensing gun 230. When the double barrel syringe 210 with the mixing tip 220 are secured within the dispensing gun 230, the composition within the double barrel syringe 210 can be applied onto a tooth surface including interproximal areas and/or subgingivally areas of a patient.

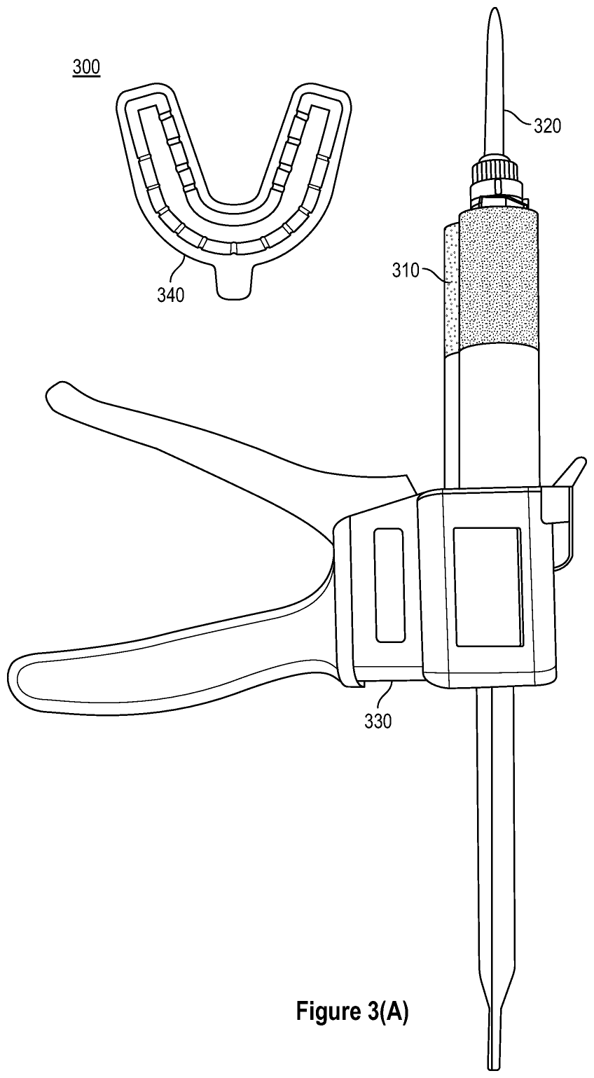

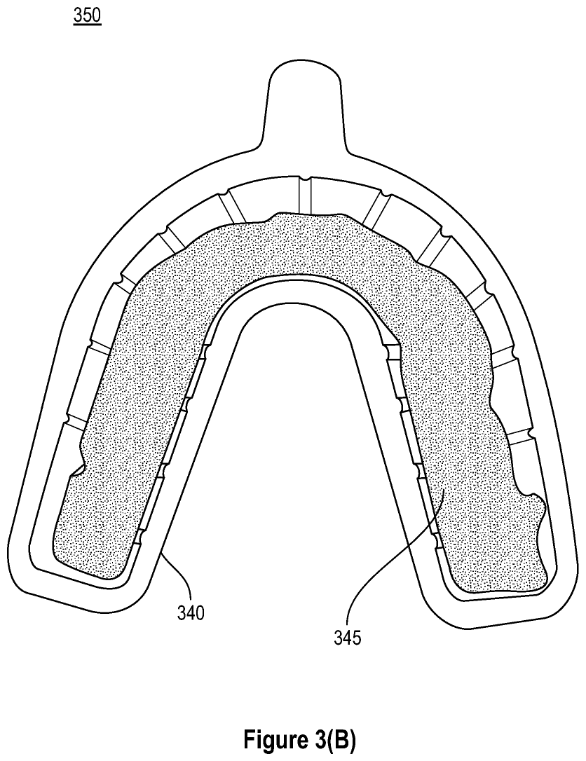

Referring to FIG. 3(A), the cleaning system 300 is shown fully assembled with the double barrel syringe 310 and mixing tip 320 configured securely within the dispensing gun 330. The composition with the double barrel syringe 310 can be dispensed through the mixing tip 320. The composition within the double barrel syringe 310 consists of a paste A. and a paste B that are to be mixed together within the mixing tip 320. The handle of the dispensing gun 330 can be pressed inward to enable the composition to be dispensed through the mixing tip 320 onto a dental tray 340. In other embodiments, the mixed composition can be disposed through the mixing tip 320 directly onto the tooth surface or dental arch. The colorization of the mixed composition within the mixing tip 320 is clearly shown within the mixing tip 320. The mixed composition will loosen the dental calculus on the tooth surface, interproximal areas, and subgingivally areas. The dental calculus will include cavities, mineralized plaque, deep stains etc. The composition will be placed on the tooth surface for a reaction time to enable the mixed composition to loosen the dental calculus. The reaction time can be anywhere from thirty to ninety seconds. In other embodiments, the reaction time can either be longer or shorter as required. After the reaction time is complete, the composition can be rinsed off of the tooth surface to begin the cleaning process. Further, for additional cleanings of the tooth surface, the composition can be reapplied onto the tooth surface through the mixing tip 320.

In FIG. 3(B), a diagram 350 of the dental tray 340 with the mixed composition 345 placed within the dental tray 340 is shown. The mixed composition 345 will be placed on the dental arch of the patient. The mixed composition will rest on the tooth surface, interproximal areas, and subgingivally areas for a reaction time. The mixed composition 345 will soften and loosen the dental calculus to make it easier to remove the dental calculus from the tooth surface, interproximal areas, and subgingivally areas. The physician or dentist can thereby place the dental tray 340 with the mixed composition 345 on the dental arch to enable the mixed composition 345 to loosen and soften the dental calculus present within the dental arch.

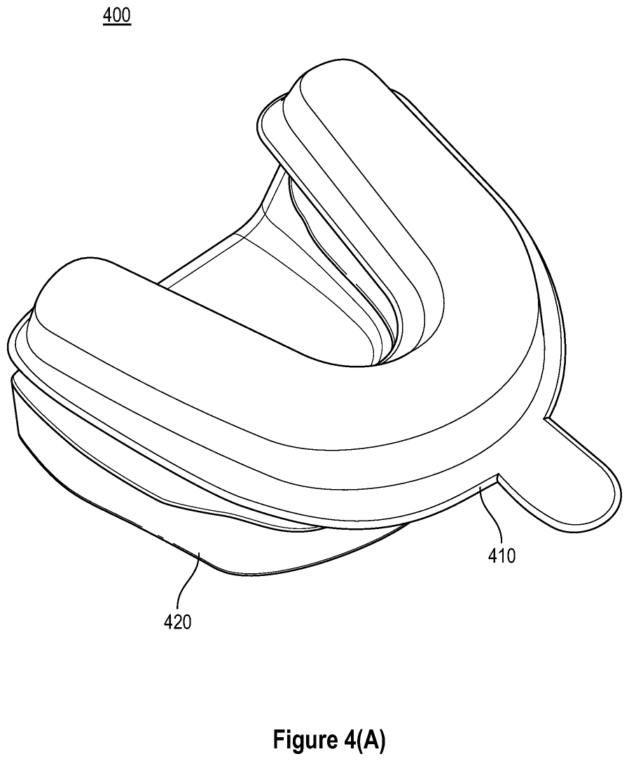

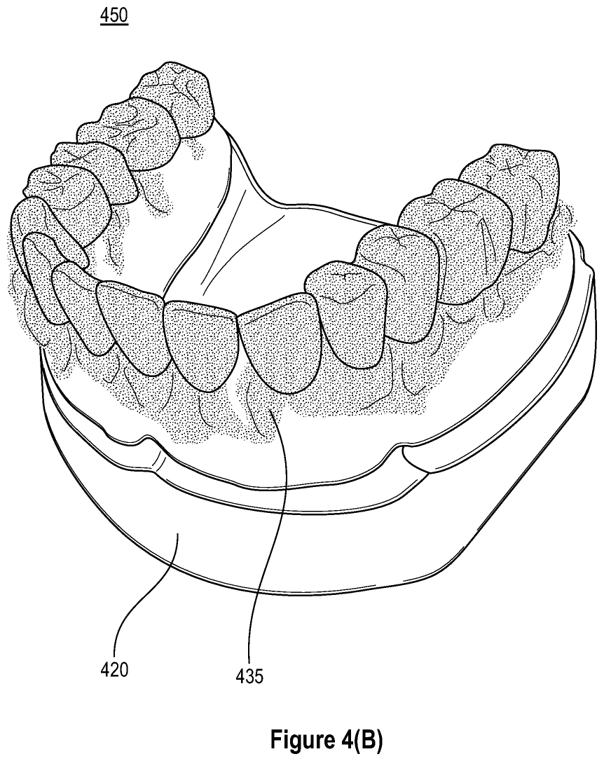

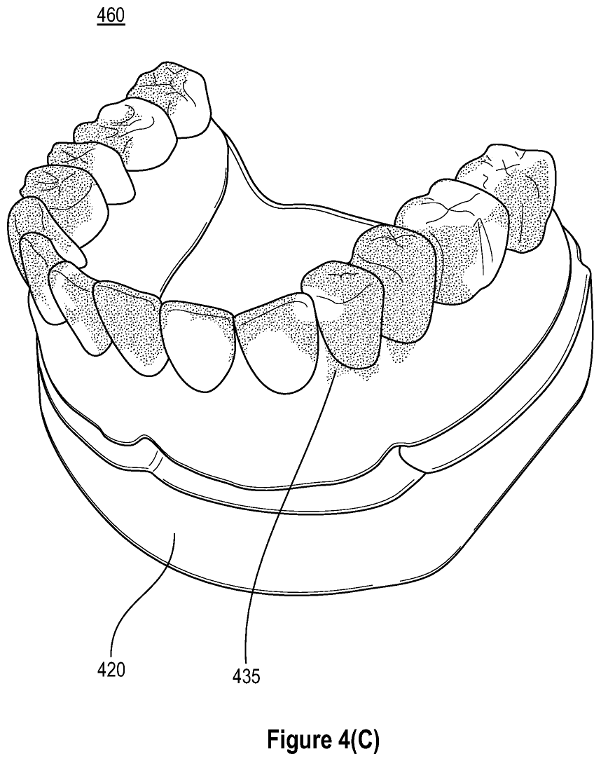

Referring to FIG. 4(A), a system. 400 in which the dental tray 410 with the mixed composition is placed on the dental arch 420 is shown. Moreover, the composition is driven into interproximal tooth areas of the tooth surface where mineralized plaque and deep stains have been formed. The composition is allowed to rest in these interproximal tooth areas to loosen and soften the dental calculus of the mineralized plaque and deep stains. The formation of a cascade of highly reactive calculus softening and tooth cleaning/whitening species are formed along the gingiva and/or sub-gingival area of the tooth surface. As such, the effervescing action of the mixed composition of paste A and paste B drives the calculus softening and/or tooth cleaning components into the areas of the mineralized plaque, cavities, deep stains, etc.

With respect to FIG. 4(A), the mixed composition is configured to aggressively attack semi-mature to mature mineralized dental calculus. The mixed composition also does not damage any tooth enamel or oral mucosa near the tooth surface. The chemical reaction of the mixed composition and the dental calculus occurs. In spite of the composition being on the dental calculus on the tooth surface, interproximal areas, and subgingivally areas, a pH buffering system is incorporated into the composition to buffer the pH to 7.0-8.0 pH. As a result, on completion of the reaction time of thirty to ninety seconds or longer, the risk of damage to tooth enamel or irritation of the oral mucosa is decreased. The mixed composition also includes a colorimetric indicator function to aid the clinician/hygienist in ensuring the proper mixing of paste A and paste B to form a seafoam green color as an additional safety feature for the composition when it is placed on the tooth surface 310.

In FIG. 4(B), the system 450 with the mixed composition 435 on the dental arch 420 is shown. The stain removing/whitening functionality of the mixed composition is accomplished by incorporating peroxide compounds to form reactive oxygen species upon the mixing of paste A and paste B. As such the removal of deep stains or dental calculus in interproximal tooth areas of the tooth surface that can be difficult to reach otherwise will occur. The dental arch 420 also receives the supplemental polishing agents present in the mixed composition 435 in the form of precipitated amorphous hydrated silicas to aid in the cleaning of the tooth surface and provide a “clean mouth feel” to the individual involved. The prevention of harm to areas of the tooth surface are also aided by the mixed composition 435 having anti-inflammatory, hemostatic, and other calmative effects. Moreover, the anti-inflammatory, hemostatic, and other calmative effects are botanical in nature. As such, the tooth surface will have a lesser likelihood of harm to the enamel, gums, or other area of the tooth surface due to the application of the mixed composition 435 on the tooth surface.

In FIG. 4(C), the cleaning process can begin when the mixed composition 435 is rinsed with water or other rinsing liquid off the tooth surface over the dental arch 420. The mixed composition 435 will be rinsed after the reaction time of thirty to ninety seconds is complete. The reaction time will allow the dental calculus to be loosened and softened. Once the mixed composition 435 is rinsed off of the tooth surface over the dental arch 420, the cleaning will begin. Further, in other embodiments, the mixed composition can be reapplied to the over the dental arch for additional cleanings when necessary.

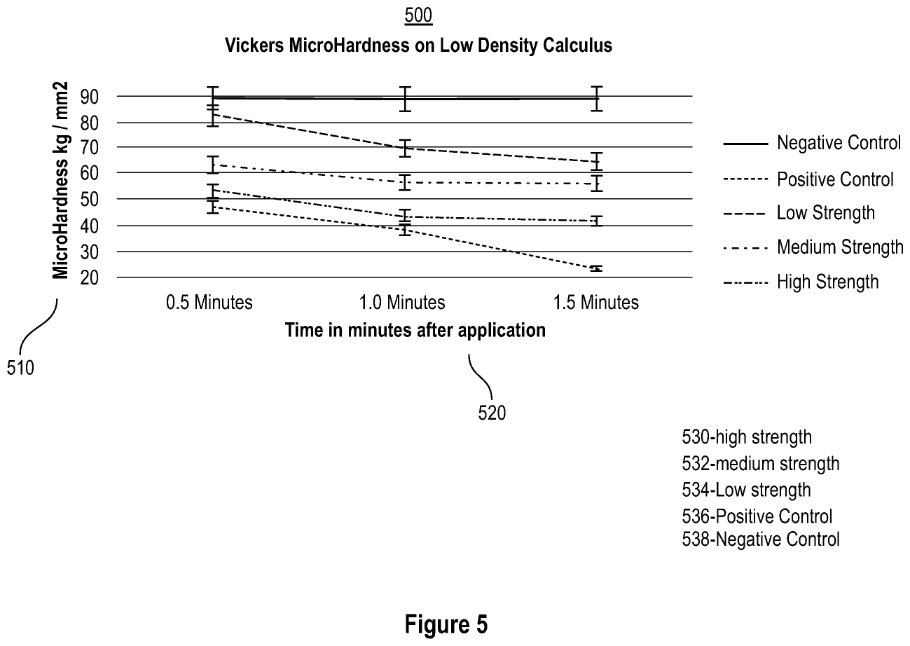

Referring to FIG. 5, a graph 500 illustrating the micro-hardness on the low density calculus on the tooth surface is illustrated. The micro-hardness 510 is illustrated along with time in minutes after application of the composition, or the reaction time 520, As shown in the comparison graph 500, the micro-hardness 510 of the dental calculus that includes deep stains and mineralized plaque will be harder when the mixed composition is first applied to the tooth surface. The reaction time can be anywhere from thirty to ninety seconds, or longer. Time is shown for 0.5 minutes or thirty seconds, 1.0 minutes or sixty seconds, and 1.5 minutes or ninety seconds.

In FIG. 5, the hardness of the dental calculus, or the micro-hardness 510 will be greater as the mixed composition is first placed on the tooth surface, and the interproximal areas along the gumline and other subgingivally areas. In addition, also illustrated are the high strength, 530, medium strength 532, low strength 534, positive control 536, and negative control 538 of the micro-hardness 510. The various strengths and controls for the micro-hardness 510 are shown for the entire reaction time 520 from 0.5 minutes to 1.5 minutes, or thirty seconds to ninety seconds. As the reaction time increases, the dental calculus on the tooth surface will loosen and soften due to the composition. As such, the low strength 534, high strength 530, medium strength 532, and positive control 536 of the micro-hardness 510 is reduced as the reaction time 520 moves from 0.5 minutes to 1.5 minutes. After 1.5 minutes or ninety seconds, the micro-hardness 510 of the dental calculus has softened and loosened considerably. Rinsing fluid such as water can then be applied to the tooth surface to rinse the tooth surface, interproximal areas, and subgingivally areas of the composition. Afterward, the tooth surface has been cleaned of much of the dental calculus that had been originally present. The composition can also be reapplied to the tooth surface, interproximal areas, and subgingivally areas for additional cleaning. As with the initial cleaning, the various strengths and controls of the micro-hardness 510 will thereby be reduced as the reaction time 520 increases from 0.5 minutes to 1.5 minutes.

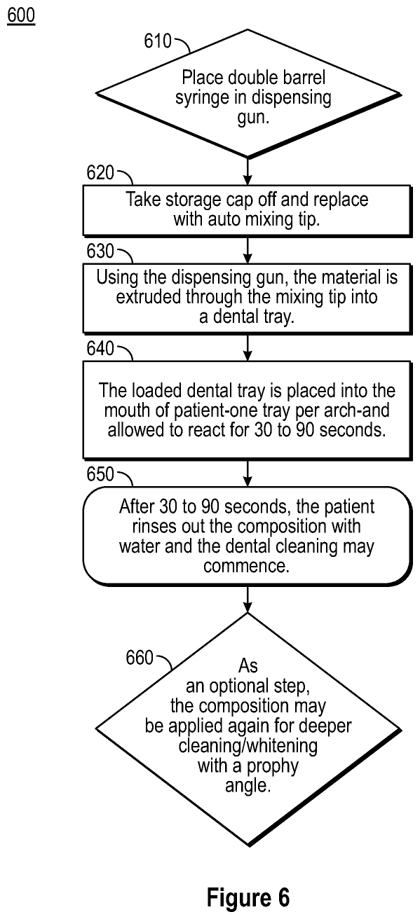

In FIG. 6, a method 600 for dispensing the mixing composition on the tooth surface is illustrated. The mixing composition can loosen the dental calculus and aid in the removal of the dental calculus on the tooth surface. The mixing composition can soften and loosen the dental calculus from a reaction time on the tooth surface, interproximal areas along a gumline, and or subgingivally areas. Rinsing fluid such as water can then rinse off the composition from the tooth surface to complete the current cleaning process.

In FIG. 6, at step 610, the double barrel syringe with paste A and paste B is placed in the dispensing gun. Initially, within the double barrel syringe, paste A and paste B are each positioned in a respective barrel within the double barrel syringe.

In FIG. 6, at step 620, the storage cap off of the double barrel syringe are taken off and replaced with an auto-mixing tip. With the auto mixing tip, the composition of paste A and Paste B can be combined into a single mixed composition that results in a seafoam green colorization. The mixed composition will loosen and soften the dental calculus on the tooth surface, interproximal areas along the gumline, and other subgingivally areas.

In FIG. 6, at step 630, the dispensing gun is used to dispose the mixed composition through the mixing tip. Paste A and paste B are completely mixed together as the composition is disposed through the mixing tip and onto the dental tray. The mixed composition can rest on the dental tray in preparation to be placed onto the tooth surface.

Referring to FIG. 6, at step 640, the loaded dental tray with the mixed composition is placed into the mouth of the patient on the tooth surface of the patient. One tray per arch is placed within the mouth of the patient. The mixed composition is placed in contact with the tooth surface or tooth region of the patient. The composition is left to react on the tooth surface for a reaction time from thirty to ninety seconds. The mixed composition also reacts with the interproximal areas along the gumline and other subgingival areas. Leaving the composition on the tooth surface for the reaction will enable the composition to loosen and soften the dental calculus on the tooth surface, gumline, and other subgingivally areas. The loosening and softening of the dental calculus will aid in the removal of the dental calculus.

With respect to FIG. 6, at step 650, after the reaction time of the thirty to ninety seconds is complete, the composition can be rinsed from the tooth surface, interproximal areas, and subgingivally areas. The patient will rinse the composition off with water, and the dental cleaning will commence.

Referring to FIG. 6, at step 660, an optimal step can occur. The mixed composition can be applied again to the tooth surface for a deeper cleaning, stain removal and whitening. If the patient or dentist so chooses, the mixed composition can be reapplied again using the steps described in steps 610-650.

Those skilled in the art will appreciate that the example embodiments are non-exhaustive and that embodiments other than that described here may be included without departing from the scope and spirit of the presently disclosed embodiments.

### Advantages

Overall, the mixed composition provides several advantages. The mixed composition can be positioned on the tooth surface over the dental arch to soften and loosen the dental calculus. The mixed composition will loosen and soften the dental calculus including hardened plague and deep stains on the tooth surface, interproximal areas and subgingival areas that are normally hard to reach.

The mixed composition being placed over the dental arch for the reaction time allows the mixed composition to react with the dental calculus on the tooth surface, interproximal areas, and subgingival areas to soften and loosen the dental calculus. The micro-hardness of the dental calculus is reduced with greater reaction time.

The rinsing of the mixed composition off of the tooth surface, interproximal areas, and subgingival areas will allow the cleaning to begin. The dental calculus can be greatly reduced over the dental arch. If any dental calculus remains, the mixed composition can be reapplied using the dispensing gun, mixing tip, and dental tray. The mixed composition can be reapplied as need to continue to remove any remaining dental calculus in and around the tooth surface, interproximal areas, and/or subgingival areas.

### CONCLUSION

All references, including granted patents and patent application publications, referred herein are incorporated herein by reference in their entirety.

All the features disclosed in this specification, including any accompanying abstract and drawings, may be replaced by alternative features serving the same, equivalent or similar purpose, unless expressly stated otherwise. Thus, unless expressly stated otherwise, each feature disclosed is one example only of a generic series of equivalent or similar features.

Various aspects of the invention have been described above by way of illustration, and the specific embodiments disclosed are not intended to limit the invention to the particular forms disclosed. The particular implementation of the system provided thereof may vary depending upon the particular context or application. The invention is thus to cover all modifications, equivalents, and alternatives falling within the spirit and scope of the following claims. It is to be further understood that not all of the disclosed embodiments in the foregoing specification will necessarily satisfy or achieve each of the objects, advantages, or improvements described in the foregoing specification.

The corresponding structures, materials, acts, and equivalents of all means or step plus function elements in the claims below are intended to include any structure, material, or act for performing the function in combination with other claimed elements as specifically claimed.

What is claimed is:

1. A method comprising:
placing a double barrel syringe into a dispensing gun;
using the dispensing gun to place a composition of paste A and paste B within the double barrel syringe through a mixing tip onto a tooth surface and interproximal areas along a gumline, and/or subgingival areas, wherein the composition softens and aids in removal of dental calculus, tartar, and/or plaque on one or more areas of the tooth surface require cleaning; and
rinsing the one or more areas of the tooth surface to enable the cleaning to begin.

2. The method of claim 1, wherein the composition includes a colorization that ensures proper mixing of paste A and paste B.

3. The method of claim 1, wherein a combination of the composition softens the dental calculus, tartar, and/or plaque in the one or more areas of the tooth surface to enable the cleaning to occur.

4. The method of claim 1, further comprising:
reapplying the composition onto the tooth surface, interproximal areas, along the gumline, and/or subgingival to provide additional cleaning of a dentition and surrounding oral tissues.

5. The method of claim 1, wherein a reaction time is provided for the tooth surface after the composition is applied to the tooth surface.

6. The method of claim 1, wherein the tooth surface is rinsed of the composition after a reaction time is provided for the tooth surface.

7. The method of claim 1, further comprising:
providing the composition through a mixing tip onto the tooth surface, and interproximal areas along the gumline, and/or the subgingival areas.

8. A method comprising:
placing composition in a double barrel syringe;
dispensing the composition through a mixing tip configured at an end of the double barrel syringe onto a tooth surface to contact unclean areas of the tooth surface to soften dental calculus, tartar, and/or plaque to enable the dental calculus to be more readily removed and enable the unclean areas of the tooth surface to be cleaned after a reaction time; and
rinsing the tooth surface after the reaction time to enable the cleaning to occur on the unclean areas of the tooth surface.

9. The method of claim 8, further comprising:
dispensing the composition through the mixing tip onto a dental tray.

10. The method of claim 8, wherein the reaction time is less than or equal to ninety seconds.

11. The method of claim 8, wherein a seafoam colorization of the composition ensures that paste A and paste B have been properly mixed to form the composition.

12. The method of claim 8, further comprising:
placing the composition onto a dental tray to be placed over a dental arch.

13. The method of claim 12, wherein reaction time provides a time to loosen/soften the dental calculus on the unclean areas of the tooth surface.

14. The method of claim 8, wherein the composition loosens/softens the dental calculus without damaging tooth enamel along the gumline or soft oral tissue.

15. A system comprising:
a double barrel placed into a dispensing gun, wherein a mixing tip is affixed to the dispensing gun;
the dispensing gun to place a composition composed of a paste A and paste B within the double barrel syringe onto a tooth surface and interproximal areas along a gumline, and/or subgingival areas, wherein the composition softens and aids in removal of dental calculus, tartar, and/or plaque on one or more areas of the tooth surface, gumline, and/or subgingival areas that require cleaning; and
the one or more areas of the tooth surface are rinsed to enable the cleaning to begin on the one or more areas of the tooth surface.

16. The system of claim 15, wherein the composition includes an alkaline phase and an acidic phase.

17. The system of claim 15, wherein the combined paste A and paste B loosens/softens the dental calculus to enable the one or more areas of the interproximal areas, the gumline, and/or the subgingival areas to be cleaned.

18. The system of claim 15, wherein a reaction time for the composition on the tooth surface is greater than or equal to thirty seconds.

19. The system of claim 15, wherein a micro-hardness of the dental calculus is reduced with increased reaction time.

20. The system of claim 15, wherein the composition is rinsed from the one or more areas of the tooth surface, interproximal areas, the gumline, and/or the subgingival areas after a reaction time of least ninety seconds.
